(19) United States
(12) Patent Application Publication
(10) Pub. No.: US 2023/0056443 A1
(43) Pub. Date: Feb. 23, 2023

(54) EQUINE GASTRIC HEALTH FORMULATION

(71) Applicant: Nathalie Boucher, Peace River (CA)

(72) Inventor: Nathalie Boucher, Peace River (CA)

(21) Appl. No.: 17/445,596

(22) Filed: Aug. 21, 2021

(51) Int. Cl. A61K 36/534; A61K 36/288; A61K 36/31; A61K 47/36

(52) U.S. Cl. CPC A61K 36/534; A61K 36/288; A61K 36/31; A61K 47/36

(57) ABSTRACT

An equine gastric health formulation that is operable to heal gastrointestinal disorders such as but not limited to ulcers, wherein the present invention further improves liver function and detoxification of the horse. The formulation of the present invention in a preferred embodiment is water based wherein fifty percent of the formulation is distilled water which is approximately five hundred milliliters. A concentration of one percent of the total solution is formed by origanum vulgare. Another two percent of the formula being represented equally is silybum marianum and taraxacum. Three drops of mentha piperita are also dispensed into the formulation of the present invention. Wherein the formulation includes ten percent of chlorophyllin and thirty seven percent is camelina oil. Additionally, four tablespoons of acacia gum powder are mixed into the formulation.

### FIELD OF THE INVENTION

The present invention relates generally to equine health products, more specifically but not by way of limitation, a formulation that is operable to improve overall health and gastrointestinal health to address conditions such as but not limited to ulcers, wherein the present invention further improves liver function and detoxification of the horse.

### BACKGROUND

Gastric or stomach ulcers are sores that form on the stomach lining. They are common in horses, with about fifty to ninety percent of horses having ulcers at some point. The ulcers can affect any horse at any age but occur most frequently in horses that perform athletic activities such as racing and showing. It has been found that exercise increases gastric acid production and decreases blood flow to the gastrointestinal tract. Furthermore, when horses exercise the acidic fluid in the stomach splashes and exposes the upper, more vulnerable portion of the stomach to an acidic pH.

The stomach of the horse is relatively smaller in comparison to other species given the size of the horse itself. As a result, horses cannot handle large amounts of food. Horses graze and eat frequent, small portions of feed for extended periods because of their gastrointestinal structure. During grazing situations, a steady flow of acid is required for digestion and as such the horse's stomach produces continuously ultimately producing up to nine gallons of acidic fluid per day. In a natural, high-roughage diet, the acid is buffered by both feed and saliva. When horses are fed two times per day, the stomach is subjected to a prolonged period without feed to neutralize the acid. In addition, high-grain diets produce volatile fatty acids that can also contribute to the development of ulcers. Physical and environmental stressors such as transport stress and stall confinement are additional risk factors. Lastly, chronic administration of some nonsteroidal anti-inflammatory drugs such as phenylbutazone can decrease the production of the protective mucus layer of the stomach, making the gastrointestinal tract more susceptible to ulcers.

The hindgut is significant to the horse's digestive process and its health. It is the home of bacterial environment that convert fiber to energy. It may also be the source of so many of the problems we are seeing. Research has shown that hindgut ulcers also exist and are quite common. Colonic ulcers predominantly affect performance horses and are frequently found in horses already suffering from gastric ulcers. The increase in digestive health issues, in the stomach and in the hindgut have evolved from how the horses are cared for and fed.

The health of the hindgut is dependent on the delicate balance between beneficial and harmful bacteria and is easily altered. Poor digestion of modern grain-based feed causes an increase in the growth of harmful bacteria and a subsequent loss of beneficial bacteria. These events can lead to the production of toxins and can reduce the pH of the hindgut, making it more acidic than it should be. This condition is known as “hindgut acidosis”. This series of events, starting with the feeding of processed feed and leading to the more acidic hindgut, causes even more beneficial bacteria to die. The harmful toxins given off under these circumstances can enter the horse's bloodstream and cause problems such as starch-induced laminitis and tying up. These toxins can also damage the tissue lining the hindgut, resulting in areas of ulceration.

It is intended within the scope of the present invention to provide a formulation that is operable to improve the health of the gastrointestinal tract and further strengthen the liver while additionally detoxifying the horse to which the present invention has been administered.

### SUMMARY OF THE INVENTION

It is the object of the present invention to provide an equine formulation operable to improve gastrointestinal health of a horse to which the present invention is administered wherein the formulation is in an herbal liquid formula.

Another object of the present invention is to provide an equine medicinal therapy provided in a liquid form that is operable to improve overall health and gastrointestinal health wherein the present invention includes in a preferred embodiment one percent of origanum vulgare.

A further object of the present invention is to provide an equine formulation operable to improve gastrointestinal health of a horse to which the present invention is administered that includes in a preferred embodiment one percent of silybum marianum.

Still another object of the present invention is to provide an equine medicinal therapy provided in a liquid form that is operable to improve overall health and gastrointestinal health wherein the present invention includes fifty percent distilled water.

An additional object of the present invention is to provide an equine formulation operable to improve gastrointestinal health of a horse to which the present invention is administered that includes one percent of taraxacum in a preferred embodiment.

Yet a further object of the present invention is to provide an equine medicinal therapy provided in a liquid form that is operable to improve overall health and gastrointestinal health wherein the formulation of the present invention in a preferred embodiment includes thirty seven percent of camelina oil.

Another object of the present invention is to a provide an equine formulation operable to improve overall health and gastrointestinal health of a horse to which the present invention is administered that includes three drops of mentha piperita.

An alternate object of the present invention is to provide an equine medicinal therapy provided in a liquid form that is operable to heal and treat gastrointestinal conditions wherein the formulation of the present invention includes approximately four tablespoons of acacia gum powder.

Another object of the present invention is to a provide an equine formulation operable to improve overall health and gastrointestinal health of a horse to which the present invention is administered that includes ten percent of chlorophyll that is three hundred milligrams in strength.

To the accomplishment of the above and related objects the present invention may be embodied in the form illustrated in the accompanying drawings. Attention is called to the fact that the drawings are illustrative only. Variations are contemplated as being a part of the present invention, limited only by the scope of the claims.

### BRIEF DESCRIPTION OF THE DRAWINGS

A more complete understanding of the present invention may be had by reference to the following Detailed Description and appended claims when taken in conjunction with the accompanying Drawings wherein:

FIG. 1 is an outline of the method of making the formulation of the present invention.

### DETAILED DESCRIPTION

Referring now to the drawings submitted herewith, wherein various elements depicted therein are not necessarily drawn to scale and wherein through the views and figures like elements are referenced with identical reference numerals, there is illustrated an equine gastrointestinal health formulation 100 constructed according to the principles of the present invention.

An embodiment of the present invention is discussed herein with reference to the figures submitted herewith. Those skilled in the art will understand that the detailed description herein with respect to these figures is for explanatory purposes and that it is contemplated within the scope of the present invention that alternative embodiments are plausible. By way of example but not by way of limitation, those having skill in the art in light of the present teachings of the present invention will recognize a plurality of alternate and suitable approaches dependent upon the needs of the particular application to implement the functionality of any given detail described herein, beyond that of the particular implementation choices in the embodiment described herein. Various modifications and embodiments are within the scope of the present invention.

It is to be further understood that the present invention is not limited to the particular methodology, materials, uses and applications described herein, as these may vary. Furthermore, it is also to be understood that the terminology used herein is used for the purpose of describing particular embodiments only, and is not intended to limit the scope of the present invention. It must be noted that as used herein and in the claims, the singular forms “a”, “an” and “the” include the plural reference unless the context clearly dictates otherwise. Thus, for example, a reference to “an element” is a reference to one or more elements and includes equivalents thereof known to those skilled in the art. All conjunctions used are to be understood in the most inclusive sense possible. Thus, the word “or” should be understood as having the definition of a logical “or” rather than that of a logical “exclusive or” unless the context clearly necessitates otherwise. Structures described herein are to be understood also to refer to functional equivalents of such structures. Language that may be construed to express approximation should be so understood unless the context clearly dictates otherwise.

References to “one embodiment”, “an embodiment”, “exemplary embodiments”, and the like may indicate that the embodiment(s) of the invention so described may include a particular feature, structure or characteristic, but not every embodiment necessarily includes the particular feature, structure or characteristic.

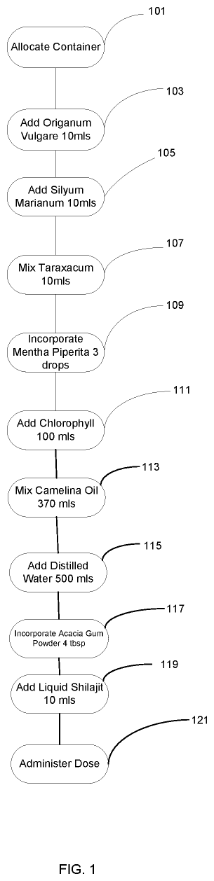

Now referring in particular to the Figures submitted as a part hereof, the equine gastrointestinal health formulation 100 and the method of manufacturing is diagrammed in FIG. 1. In step 101, a container is allocated wherein the container will have sufficient volume to accommodate all of the ingredients in the equine gastrointestinal health formulation 100. Step 103, ten milliliters of origanum vulgare, in particular a natural wild version thereof is measured and placed in the container. In a preferred embodiment of the present invention it is contemplated that the origanum vulgare will comprise one percent of the total solution of the formulation. However, while the aforementioned is the preferred percentage of origanum vulgare, it is contemplated within the scope of the present invention that the percentage of origanum vulgare could range from one half of a percent to two percent of the total solution of the present invention. In step 105, ten milliliters of silybum marianum(non-alcoholic) is measure and deposited into the container. In a preferred embodiment of the present invention it is contemplated that the silybum marianum will comprise one percent of the total solution of the formulation. However, while the aforementioned is the preferred percentage of silybum marianum, it is contemplated within the scope of the present invention that the percentage of silybum marianum could range from one half of a percent to two percent of the total solution of the present invention.

Step 107, ten milliliters of taraxacum is measured and dispensed into the container. In a preferred embodiment of the present invention it is contemplated that the taraxacum will comprise one percent of the total solution of the formulation. However, while the aforementioned is the preferred percentage of taraxacum, it is contemplated within the scope of the present invention that the percentage of taraxacum could range from one half of a percent to two percent of the total solution of the present invention. In step 109, three drops of menthe piperita are added to the container utilizing a suitable dropper. Within the scope of the present invention it should be understood that each drop is approximately 0.05 ml in volume. It should be understood within the scope of the present invention that a range of two to five drops of menthe piperita could be utilized in the formulation.

In step 111, one hundred milliliters of chlorophyll is added to the container. In a preferred embodiment of the present invention it is contemplated that the chlorophyll will comprise ten percent of the total solution of the formulation. However, while the aforementioned is the preferred percentage of chlorophyll, it is contemplated within the scope of the present invention that the percentage of chlorophyll could range from eight to twelve percent of the total solution of the present invention. The cholorphyll utilized is 300 mg strength/15 mls. Step 113, three hundred and seventy milliliters of camelina oil are added to the container. In a preferred embodiment of the present invention it is contemplated that the camelina oil will comprise thirty-seven percent of the total solution of the formulation. However, while the aforementioned is the preferred percentage of camelina oil, it is contemplated within the scope of the present invention that the percentage of camelina oil could range from thirty five percent to forty five percent of the total solution of the present invention.

In step 115, five hundred milliliters of distilled water is added to the container. In the preferred embodiment of the present invention the distilled water is fifty percent of the formulation of the present invention. Step 117, approximately four tablespoons of acacia gum powder is added to the container wherein the acacia gum powder is pre-hydrated. In a preferred embodiment of the present invention the preferred dose of acacia gum powder is four tablespoons it is contemplated within the scope of the present invention that the acacia gum powder could range from three to five tablespoons. Step 119, a horse will be administered twenty milliliters. While twenty milliliters is preferred it is contemplated within the scope of the present invention that a horse could be administered between fifteen and twenty five milliliters. While not illustrated in the Figure submitted herewith, it is further contemplated within the scope of the present invention that the equine gastrointestinal health formulation 100 could further include liquid shilajit. While good results have been achieved by utilizing ten milliliters of shilajit, it is contemplated within the scope of the present invention that alternate amounts could be utilized.

In the preceding detailed description, reference has been made to the accompanying drawings that form a part hereof, and in which are shown by way of illustration specific embodiments in which the invention may be practiced. These embodiments, and certain variants thereof, have been described in sufficient detail to enable those skilled in the art to practice the invention. It is to be understood that other suitable embodiments may be utilized and that logical changes may be made without departing from the spirit or scope of the invention. The description may omit certain information known to those skilled in the art. The preceding description is, therefore, not intended to be limited to the specific forms set forth herein, but on the contrary, it is intended to cover such alternatives, modifications, and equivalents, as can be reasonably included within the spirit and scope of the invention.

What is claimed is:

1. An equine gastric health formulation comprising:
a portion of origanum vulgare, wherein the portion of the origanum vulgare ranges from one half of a percent to two percent of the formulation;
a portion of silybum marianum;
a portion of taraxacum, wherein the taracum is non-alcoholic;
a portion of mentha piperita;
a portion of chlorophyll, wherein the chlorophyll is approximately ten percent of the total formulation;
a portion of camelina oil; and
an amount of distilled water, wherein the amount of distilled water is approximately fifty percent of the total volume of the formulation.

2. The equine gastric health formulation as recited in claim 1, wherein the formulation further includes approximately four tablespoons of acacia gum powder.

3. The equine gastric health formulation as recited in claim 2, wherein the portion of the silybum marianum ranges from one half of a percent to two percent of the formulation.

4. The equine gastric health formulation as recited in claim 3, wherein the portion of the camelina oil ranges from thirty five percent to forty percent of the formulation.

5. The equine gastric health formulation as recited in claim 4, wherein the amount of mentha piperita is three drops.

6. The equine gastric health formulation as recited in claim 5, wherein the volume of distilled water is five hundred milliliters.

7. An equine gastric health formulation operable to improve gastrointestinal health health of horses wherein the equine gastric health formulation comprises:
a portion of origanum vulgare, wherein the portion of the origanum vulgare is one percent of the formulation;
a portion of silybum marianum, wherein the portion of the silybum marianum is one percent of the formulation;
a portion of taraxacum, wherein the taracum is non-alcoholic, wherein the portion of the silybum marianum is one percent of the formulation;
a portion of mentha piperita, wherein the amount of menthe piperita is three drops;
a portion of chlorophyll, wherein the chlorophyllin is ten percent of the total formulation;
a portion of camelina oil, wherein the amount of camelina oil is three hundred and seventy milliliters;
an amount of distilled water, wherein the amount of distilled water is five hundred milliliters; and
four tablespoons of acacia gum powder.
